(19) United States
(12) Patent Application Publication
(10) Pub. No.: US 2015/0374337 A1
(43) Pub. Date: Dec. 31, 2015

(54) ULTRASOUND DIAGNOSTIC APPARATUS

(71) Applicants: Kabushiki Kaisha Toshiba, Minato-ku (JP); Toshiba Medical Systems Corporation, Otawara-shi (JP)

(72) Inventors: Kuramitsu NISHIHARA, Otawara-shi (JP); Akihiro KAKEE, Otawara-shi (JP); Takuya SASAKI, Otawara-shi (JP)

(73) Assignees: Kabushiki Kaisha Toshiba, Minato-ku (JP); Toshiba Medical Systems Corporation, Otawara-shi (JP)

(21) Appl. No.: 14/846,094

(22) Filed: Sep. 4, 2015

Related U.S. Application Data
(63) Continuation of application No. PCT/JP2014/052943, filed on Feb. 7, 2014.

(30) Foreign Application Priority Data
Mar. 6, 2013 (JP) 2013-044675

(51) Int. Cl. A61B 8/00

(52) U.S. Cl. CPC A61B 8/464; A61B 8/469

(57) ABSTRACT

An ultrasound diagnostic apparatus according to an embodiment includes transmitting and receiving circuitry, adding circuitry, and image generating circuitry, and control circuitry. The transmitting and receiving circuitry performs, more than once on the same scan line, an ultrasound transmission and reception repeatedly performed with phase polarities being inverted on the same scan line, according to a number set as a scan condition parameter. The adding circuitry adds together reflected wave data received as a result of the ultrasound transmission and receptions. The image generating circuitry generates an image by using the reflected wave data that have been added together. The control circuitry controls the transmitting and receiving circuitry, based on a relation between the number of the ultrasound transmission and receptions and a scan condition parameter other than the number.

### CROSS-REFERENCE TO RELATED APPLICATIONS

This application is a continuation of PCT international application Ser. No. PCT/JP2014/052943 filed on Feb. 7, 2014 which designates the United States, incorporated herein by reference, and which claims the benefit of priority from Japanese Patent Application No. 2013-044675, filed on Mar. 6, 2013, the entire contents of which are incorporated herein by reference.

### FIELD

Embodiments described herein relate generally to an ultrasound diagnostic apparatus.

### BACKGROUND

An ultrasound diagnostic apparatus performs imaging of a tissue in a subject by transmitting ultrasound pulses to the subject, receiving the reflected waves, and applying a pulse reflection method to the received reflected waves.

Generally, in the ultrasound diagnostic apparatus, scanning is performed with scan conditions having been set, the scan conditions including a scan range, a scan line density, and a frame rate. A scan range is a width of an area scanned by the ultrasound pulses, and is also called a field width, a field angle, or the like. A scan line density is the number of scan lines per unit area, and a frame rate is the number of frames per unit time. Since these parameters have trade-off relations among them, an operator operates the ultrasound probe while adjusting settings of the parameters as appropriate, according to the object of the examination, and the like.

### BRIEF DESCRIPTION OF THE DRAWINGS

FIG. 1 is a functional block diagram of an ultrasound diagnostic apparatus according to a first embodiment;

FIG. 2 is an external view of the ultrasound diagnostic apparatus according to the first embodiment;

FIG. 3 is a diagram illustrating an operating device in the first embodiment;

FIG. 4 is a diagram for description of ultrasound transmission and receptions in the first embodiment;

FIG. 5 is a diagram illustrating an index control table in the first embodiment;

FIG. 6 is a diagram illustrating an operation user interface (UI) for parameter control in the first embodiment;

FIG. 7 is a diagram illustrating a processing sequence of the parameter control in the first embodiment;

FIGS. 8A to 8D are diagrams illustrating parameter marks in the first embodiment;

FIGS. 9A to 9D are diagrams illustrating parameter marks in a modification of the first embodiment;

FIGS. 10A to 10D are diagrams illustrating parameter marks in the modification of the first embodiment;

FIG. 11 is a diagram illustrating an index control table in the modification of the first embodiment;

FIGS. 12A to 12D are diagrams illustrating parameter marks in the modification of the first embodiment;

FIGS. 13A and 13B are diagrams illustrating an operation UI with a priority mode switch in a second embodiment;

FIGS. 14A and 14B are diagrams illustrating an operation UI with a sensitivity ON/OFF switch in a third embodiment;

FIGS. 15A and 15B are diagrams for description of specification of a region of interest (ROI) in a fourth embodiment; and

FIG. 16 is a diagram illustrating a processing sequence of parameter control in the fourth embodiment.

### DETAILED DESCRIPTION

Hereinafter, ultrasound diagnostic apparatuses according to embodiments will be described with reference to the drawings. The embodiments are not limited to the following embodiments. Further, what will be described in each of the embodiments is, in principle, similarly applicable to the other embodiments.

An ultrasound diagnostic apparatus according to an embodiment includes transmitting and receiving circuitry, adding circuitry, and image generating circuitry, and control circuitry. The transmitting and receiving circuitry performs, more than once on the same scan line, an ultrasound transmission and reception repeatedly performed with phase polarities being inverted on the same scan line, according to a number set as a scan condition parameter. The adding circuitry adds together reflected wave data received as a result of the ultrasound transmission and receptions. The image generating circuitry generates an image by using the reflected wave data that have been added together. The control circuitry controls the transmitting and receiving circuitry, based on a relation between the number of the ultrasound transmission and receptions and a scan condition parameter other than the number.

### First Embodiment

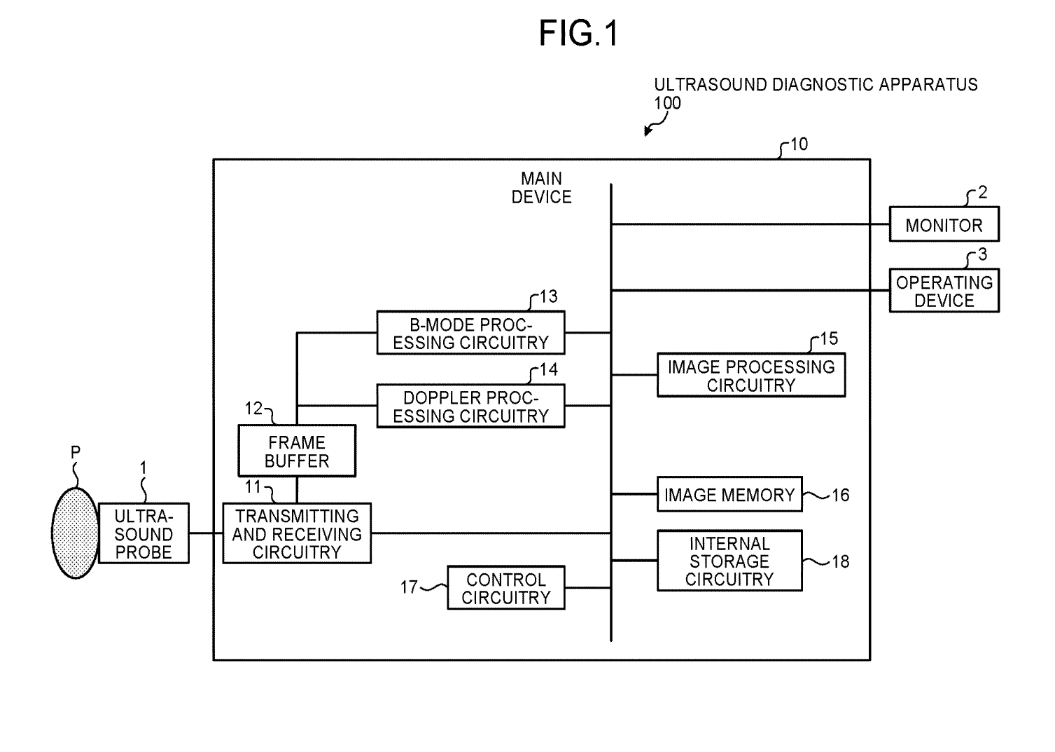

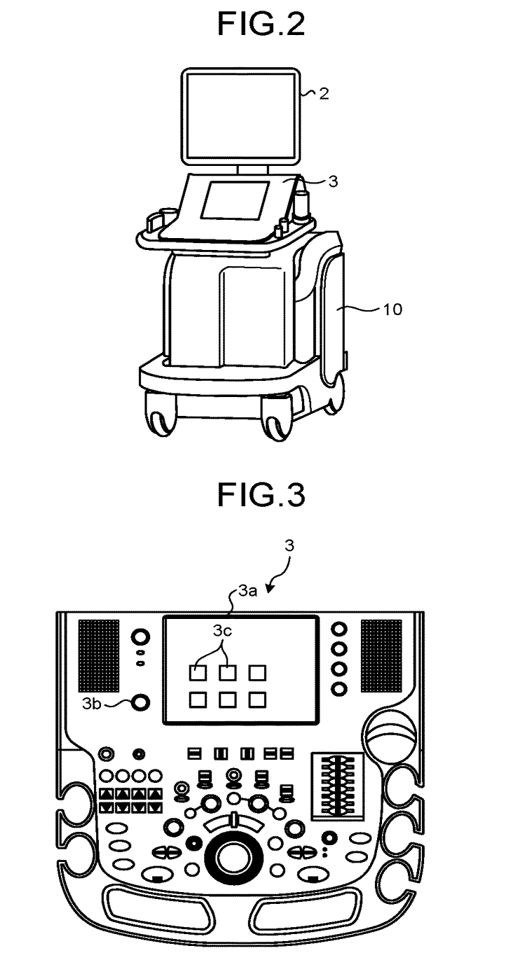

First, a first embodiment will be described. FIG. 1 is a functional block diagram of an ultrasound diagnostic apparatus 100 according to the first embodiment, and FIG. 2 is an external view of the ultrasound diagnostic apparatus 100 according to the first embodiment. As illustrated in FIG. 1 and FIG. 2, the ultrasound diagnostic apparatus 100 according to the first embodiment includes an ultrasound probe 1, a monitor 2, an operating device 3, and a main device 10.

The ultrasound probe 1 has plural piezoelectric transducer elements. The plural piezoelectric transducer elements generate, based on drive signals supplied from transmitting and receiving circuitry 11 that the main device 10 has, ultrasound pulses, receive reflected waves from a subject P, and convert the reflected waves into electric signals. Further, the ultrasound probe 1 has a matching layer provided on the piezoelectric transducer elements, a backing material that prevents propagation of ultrasound waves backward from the piezoelectric transducer elements, and the like.

When ultrasound pulses are transmitted from the ultrasound probe 1 to the subject P, the transmitted ultrasound pulses are successively reflected by an acoustic impedance discontinuous surface in a body tissue of the subject P, and are received, as echo signals, by the plural piezoelectric transducer elements that the ultrasound probe 1 has. Amplitude of the received echo signals depends on acoustic impedance difference in the discontinuous surface, by which the ultrasound pulses are reflected. If the transmitted ultrasound waves are reflected by moving blood flow, a surface of a cardiac wall, or the like, frequency of the echo signals is shifted dependently on velocity components of the moving body with respect to a direction in which the ultrasound waves are transmitted, due to the Doppler shift.

The monitor 2 displays an ultrasound image and the like generated in the main device 10.

The operating device 3 displays an operation user interface (UI) for an operator of the ultrasound diagnostic apparatus 100 to set scan condition parameters and to input other various instructions. Further, the operating device 3 receives, from the operator of the ultrasound diagnostic apparatus 100, settings of the scan condition parameters and the other various instructions, and transfers the received settings and various instructions to the main device 10.

FIG. 3 is a diagram illustrating the operating device 3 in the first embodiment. As illustrated in FIG. 3, in the operating device 3, a touch command screen (TCS) 3a, and hardware operating devices are arranged. The hardware operating devices are, for example, a track ball, a changeover switch, a button switch, a toggle switch, and the like. For example, the operator performs setting of the scan condition parameters, by operating a hardware operating device (in FIG. 3, the hardware operating device being a button switch 3b), to which a function linked with an operation UI 3c is assigned, while looking at the operation UI 3c displayed on the TCS 3a. Further, the TCS 3a displays a software switch as one of operation UIs and is able to receive an input via a contact on the software switch. In this case, for example, the operator performs setting of the scan condition parameters by directly touching the operation UI 3c displayed on the TCS 3a. To the software switches and hardware operating devices, functions are assigned by the operator, a service man, or the like.

The operating device 3 illustrated in FIG. 3 is just an example, and the embodiment is not limited to this example. Design of the whole operating device 3, arrangement of the TCS 3a and other hardware operating devices, and the like may be arbitrarily modified. Further, the operating device 3 may include another operating device, such as a keyboard, a pedal switch, or the like, which is not illustrated.

Returning to FIG. 1, the main device 10 generates, based on the reflected waves received by the ultrasound probe 1, an ultrasound image. The main device 10 has, as illustrated in FIG. 1, the transmitting and receiving circuitry 11, a frame buffer 12, B-mode processing circuitry 13, Doppler processing circuitry 14, image processing circuitry 15, an image memory 16, control circuitry 17, and internal storage circuitry 18.

The transmitting and receiving circuitry 11 has a trigger generating circuit, a transmission delay circuit, and a pulser circuit, and provides drive signals to the ultrasound probe 1. The pulser circuit repeatedly generates a rate pulse for forming ultrasound pulses of a predetermined pulse repetition frequency (PRF). The PRF is also called a rate frequency. Further, the transmission delay circuit converges the ultrasound pulses generated by the ultrasound probe 1 into a beam form, and adds, to each of the rate pulses generated by the pulser circuit, a transmission delay time that is required in determining transmission directivity for each of the piezoelectric transducer elements. Further, the trigger generating circuit applies the drive signal (drive pulse) to the ultrasound probe 1 at a timing based on the rate pulse. That is, the transmission delay circuit arbitrarily adjusts a transmission direction from a piezoelectric transducer element surface, by changing the transmission delay time added to each rate pulse.

The transmitting and receiving circuitry 11 has a function that is able to instantaneously change transmission frequency, transmission drive voltage, and the like, in order to execute, based on an instruction by the control circuitry 17, a predetermined scan sequence. In particular, changing the transmission drive voltage is realized by a linear amplifier type transmitting circuit that is able to switch over the value instantaneously, or a mechanism that electrically switches over plural power supply units.

Further, the transmitting and receiving circuitry 11 has an amplifier circuit, an analog/digital (A/D) converter, a reception delay circuit, an adder, and a quadrature detection circuit, performs various processes with respect to reflected wave signals received by the ultrasound probe 1, and generates reflected wave data. The amplifier circuit performs gain correction processing by amplifying the reflected wave signals for each channel. The A/D converter performs A/D conversion on the reflected wave signals that have been gain corrected. The reception delay circuit adds, to the digital data, a reception delay time required in determining reception directivity. The adder performs addition processing of the reflected wave signals added with the reception delay time by the reception delay circuit. By the addition processing of the adder, reflection components from a direction corresponding to the reception directivity of the reflected wave signals are emphasized. The quadrature detection circuit converts an output signal of the adder into an in-phase signal (I signal) and a quadrature-phase signal (Q-signal) of a baseband. The quadrature detection circuit stores the I signal and Q signal (hereinafter, referred to as “IQ signal”) as reflected wave data into a frame buffer 12 downstream therefrom. The quadrature detection circuit may convert the output signal from the adder into a radio frequency (RF) signal and store the RF signal into the frame buffer 12.

The B-mode processing circuitry 13 receives the reflected wave data from the transmitting and receiving circuitry 11, and generates data (B-mode data) expressing signal intensity in brightness of luminance by performing logarithmic amplification, envelope detection processing, and the like.

The Doppler processing circuitry 14 performs frequency analysis on velocity information from the reflected wave data received from the transmitting and receiving circuitry 11, extracts blood flow, tissue, and contrast agent echo components due to the Doppler shift, and generates data (Doppler data) resulting from extraction of moving object information such as an average velocity, a distribution, and power at multiple points.

The image processing circuitry 15 generates an ultrasound image from the B-mode data generated by the B-mode processing circuitry 13 and the Doppler data generated by the Doppler processing circuitry 14. Specifically, the image processing circuitry 15 generates a B-mode image from the B-mode data and generates a Doppler image from the Doppler data. Further, the image processing circuitry 15 converts a scan line signal row of an ultrasound scan into a scan line signal row of a video format represented by television or the like, to generate the ultrasound image (B-mode image and Doppler image) as a display image.

The image memory 16 is a memory that stores therein the ultrasound image generated by the image processing circuitry 15 and an image generated by image processing of the ultrasound image. For example, after a diagnosis, an operator is able to call images recorded during the examination, and still image playback or moving image playback by use of plural images is possible. Further, the image memory 16 stores therein image luminance signals that have passed the transmitting and receiving circuitry 11, other raw data, image data obtained via a network, and the like, as necessary.

The control circuitry 17 controls the overall processing in the ultrasound diagnostic apparatus 100. Specifically, the control circuitry 17 controls, based on the settings of the scan condition parameters and various instructions, which have been input by the operator via the operating device 3, and various programs and various types of setting information loaded from the internal storage circuitry 18, the processing of the transmitting and receiving circuitry 11, the B-mode processing circuitry 13, the Doppler processing circuitry 14, and the image processing circuitry 15, and performs control such that the ultrasound image or the like stored in the image memory 16 is displayed on the monitor 2.

The internal storage circuitry 18 stores therein: device control programs for performing ultrasound transmission and reception, image processing, and display processing; various data, such as diagnostic information (for example, patient IDs, findings by medical doctors, and the like), diagnostic protocols, and various types of setting information; and the like. Further, the internal storage circuitry 18 may also be used, as necessary, for storage of images stored in the image memory 16, and the like.

The transmitting and receiving circuitry 11 and the like built in the main device 10 may be configured of hardware, such as an integrated circuit, but may also be a program modularized by software.

The control circuitry 17 is able to cause the transmitting and receiving circuitry 11 to operate in a mode (hereinafter, harmonic imaging mode) for imaging harmonic components, by controlling the transmitting and receiving circuitry 11. Further, in the harmonic imaging mode, a technique (hereinafter, polarity inversion technique) for offsetting fundamental wave components by inverting the phase polarity of ultrasound beams is used.

The polarity inversion technique is a technique for offsetting fundamental wave components included in reflected wave signals and extracting harmonic components, by performing an ultrasound transmission and reception (a transmission of ultrasound beams and a reception of the reflected wave signals) twice on the same scan line. For example, in the first transmission, the phase polarity of ultrasound beams is made positive, and in the second transmission, the phase polarity is inverted from the first phase polarity to be negative. By adding together the reflected wave signals obtained by the two transmission and receptions, since the phases of their fundamental components are opposite to each other, the fundamental components are offset by each other, but since the phases of their harmonic components generated during ultrasound wave propagation match each other, the harmonic components are enhanced.

The control circuitry 17 controls the transmitting and receiving circuitry 11 such that, on the same scan line, one set or plural sets of the twice-in-a-set ultrasound transmission and reception/receptions repeatedly performed with the phase polarities inverted on the same scan line is/are performed.

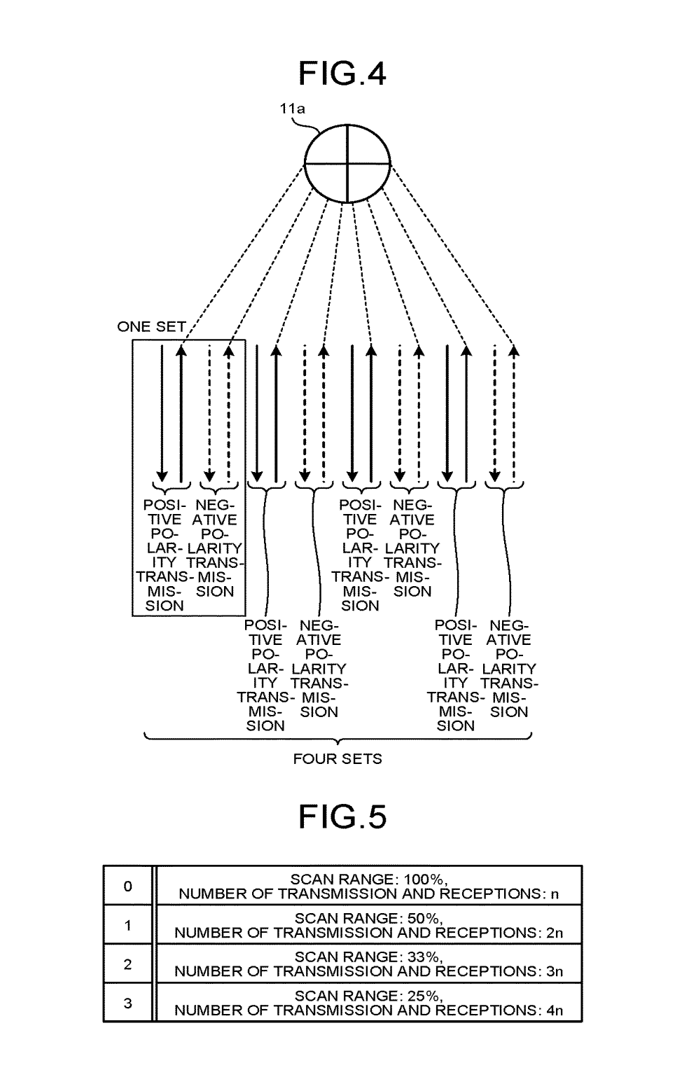

FIG. 4 is a diagram for description of the ultrasound transmission and receptions in the first embodiment. As illustrated in FIG. 4, an ultrasound transmission and reception (a downward solid-lined arrow indicating the transmission and an upward solid-lined arrow indicating the reception) performed with a positive polarity and an ultrasound transmission and reception (a downward broken-lined arrow indicating the transmission and an upward broken-lined arrow indicating the reception) performed with a negative polarity are one set of ultrasound transmission and receptions. For example, the transmitting and receiving circuitry 11 performs, under the control by the control circuitry 17, as illustrated in FIG. 4, four sets of the twice-in-a-set ultrasound transmission and receptions.

When plural sets of the ultrasound transmission and receptions are performed, as illustrated in FIG. 4, an adder 11a that the transmitting and receiving circuitry 11 has adds together reflected wave data corresponding to the plural sets received as a result of the ultrasound transmission and receptions. For example, the adder 11a performs the addition of the reflected wave signals with RF signals or IQ signals. Further, the image processing circuitry 15 generates an image by using the reflected wave data corresponding to the plural sets that have been added together. That is, the reflected wave data corresponding to one scan line used by the image processing circuitry 15 in generating the image is the sum of the reflected wave data corresponding to the plural sets.

In this case, the harmonic components as signals used in the generation of the image linearly increase (for example, are doubled) according to the number of sets of transmission and receptions, but since appearance of noise components is stochastically random, the noise components do not necessarily increase linearly (for example, they become √2 times as much). As a result, as compared to a normal case where one set of the twice-in-a-set ultrasound transmission and reception is performed, the S/N ratio of the whole image including a deep part is improved by 2/√2=√2 times. For example, when four sets of the twice-in-a-set ultrasonic transmission and receptions are performed, the S/N ratio theoretically increases by 6 dB. As described above, when plural sets of the ultrasound transmission and receptions are performed, the sensitivity is able to be improved.

Although the description has been made by illustration of the four sets in FIG. 4, the embodiment is not limited thereto, and the ultrasound diagnostic apparatus 100 is able to perform any number of sets of the twice-in-a-set ultrasound transmission and receptions. For example, if two sets of the twice-in-a-set ultrasonic transmission and receptions are performed, the S/N ratio theoretically increases by 3 dB, and if eight sets of the twice-in-a-set ultrasonic transmission and receptions are performed, the S/N ratio theoretically increases by 9 dB.

The control circuitry 17 according to the first embodiment receives settings of the scan condition parameters (hereinafter, “parameters”) via the operating device 3 and controls the transmitting and receiving circuitry 11 according to the received parameters. In the first embodiment, the parameters include “number of transmission and receptions”, in addition to “scan range”, “scan line density”, and “frame rate”. The “number of transmission and receptions” is the number of times the ultrasound transmission and reception is performed on the same scan line. For example, if “twice” is set as the number of transmission and receptions, the transmitting and receiving circuitry 11 performs one set of the twice-in-a-set ultrasound transmission and reception. Further, for example, if “eight times” is set as the number of transmission and receptions, the transmitting and receiving circuitry 11 performs four sets of the twice-in-a-set ultrasound transmission and receptions. Naming of the parameters and the like is able to be arbitrarily modified.

These four parameters have trade-off relations among them. That is, for example, if “scan range” is widened in order to widen the field width and the other parameters are fixed, “scan line density” has to be decreased correspondingly thereto and thus spatial resolution is reduced. Further, similarly, if “scan range” is widened and the other parameters are fixed, “frame rate” has to be decreased correspondingly thereto, and thus time resolution is reduced. Further, similarly, if “scan range” is widened and the other parameters are fixed, “number of transmission and receptions” has to be decreased correspondingly thereto and thus sensitivity is reduced. The trade-off relations hold true similarly among three parameters and among four parameters.

Hereinafter, an example will be described, in which the control circuitry 17 provides an operation UI realizing a trade-off between “scan range” and “number of transmission and receptions” with “scan line density” and “frame rate” being fixed for the four parameters and controls ultrasound transmission and receptions according to settings received from an operator.

As described above, “number of transmission and receptions” is the number of ultrasound transmission and receptions performed on the same scan line. Therefore, if “number of transmission and receptions” is increased, a collecting time for reflected wave data required in generation of data of one ultrasound image is increased correspondingly to the increase in that number and thus time resolution is reduced. Therefore, hereinafter, in order to improve the sensitivity with the time resolution being maintained, the control circuitry 17 narrows “scan range” by the rate of increase in the number of ultrasound transmission and receptions.

When one hundred scan lines are assumed to be required in generating data for one ultrasound image and “number of transmission and receptions” is “twice”, the number of transmission and receptions required to generate the data of one ultrasound image is 100 (scan lines)×2 (the number of transmission and receptions per scan line)=200 times. If “number of transmission and receptions” is doubled to “four times”, the number of transmission and receptions required to generate the data of one ultrasound image is 100 (scan lines)×4 (the number of transmission and receptions per scan line)=400 times.

Thus, in the first embodiment, while maintaining “scan line density” and “frame rate”, in order to absorb the Increase in “number of transmission and receptions”, the control circuitry 17 controls “scan range”. Specifically, the control circuitry 17 causes the “scan range” to be “½”. In this case, since the number of scan lines required in order to generate the data of one ultrasound image is fifty scan lines, the number of transmission and receptions required in order to generate the data of one ultrasound Image is 50 (scan lines)×4 (the number of transmission and receptions per scan line)=200 times, and thus with “scan line density” and “frame rate” being maintained, the sensitivity is able to be improved.

Similarly, if “number of transmission and receptions” is “eight times”, the control circuitry 17 causes “scan range” to be “¼”. In this case, since the number of scan lines required in order to generate the data of one ultrasound image is twenty five scan lines, the number of transmission and receptions required in order to generate the data of one ultrasound image is 25 (scan lines)×8 (the number of transmission and receptions per scan line)=200 times, and as a result, with “scan line density” and “frame rate” being maintained, the sensitivity is able to be improved.

In contrast, instead of antecedently changing the parameter, “number of transmission and receptions”, the parameter, “scan range”, may be antecedently changed. In this case, the control circuitry 17 controls “number of transmission and receptions” in order to maintain “scan line density” and “frame rate”. For example, if “scan range” is “½”, the control circuitry 17 causes “number of transmission and receptions” to be “four times”.

In the first embodiment, the control circuitry 17 realizes such adjustment of the trade-off between “scan range” and “number of transmission and receptions” by providing an operation UI for index control. FIG. 5 is a diagram illustrating an index control table in the first embodiment. For example, the control circuitry 17 stores the index control table illustrated in FIG. 5. This index control table may be set upon shipment of the ultrasound diagnostic apparatus 100 or may be able to be set and edited by an operator. As illustrated in FIG. 5, for example, in the index control table, combinations of parameter values of “scan range” and “number of transmission and receptions” are listed correspondingly with indices. For example, correspondingly with an index, “0”, “scan range: 100%” and “number of transmission and receptions: n” are stored. Herein, n=“2”. In the index control table, further, correspondingly with each of indices, “1”, “2”, and “3”, “scan range” and “number of transmission and receptions” are stored.

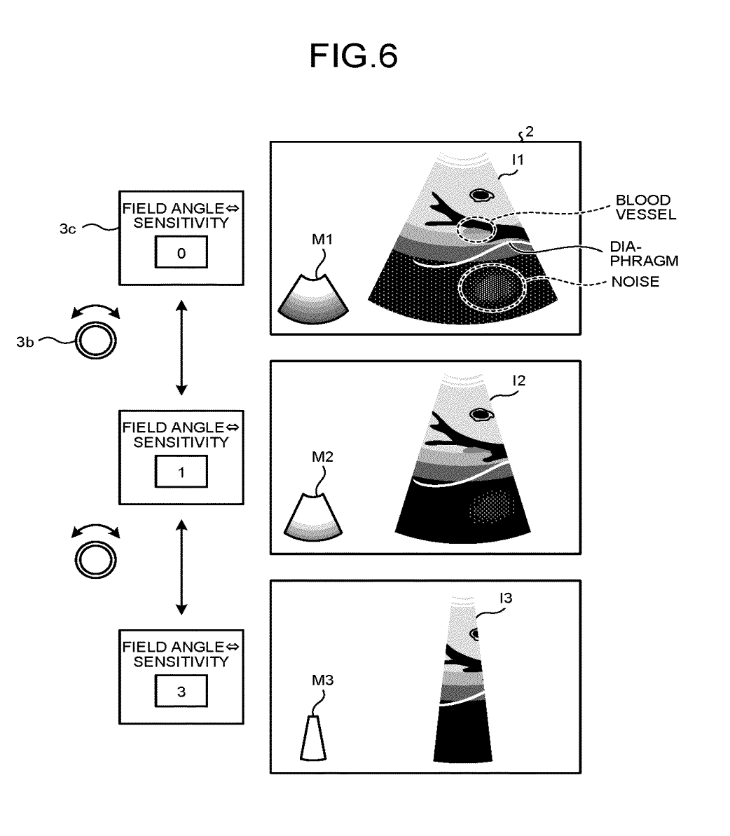

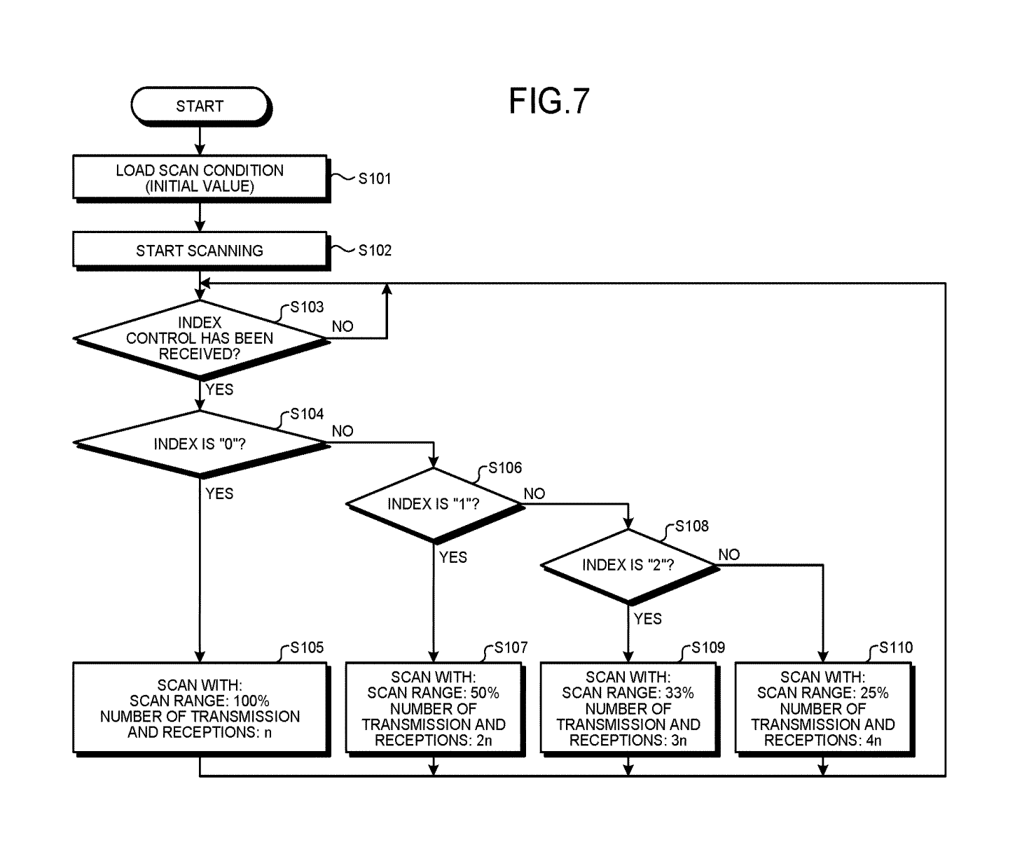

FIG. 6 is a diagram illustrating an operation UI for parameter control in the first embodiment, and FIG. 7 is a diagram illustrating a processing sequence of the parameter control in the first embodiment. For example, the control circuitry 17 displays the operation UI 3c illustrated in FIG. 6 on the TCS 3a. On this operation UI 3c, in order to indicate that the operation UI 3c is an operation UI realizing the trade-off between “scan range” and “number of transmission and receptions”, for example, a name, “Field Angle [symbol] Sensitivity” is displayed. The number (for example, “0”) in a rectangle in the operation UI 3c corresponds to the index illustrated in FIG. 5. The function of this operation UI 3c is assigned to the button switch 3b beforehand, and the operator is able to switch over the number in the rectangle among “0” [symbol] “1” [symbol] “2” [symbol] “3” by rotationally operating the button switch 3b from side to side.

First, according to description of this processing sequence, as illustrated in FIG. 7, the control circuitry 17 loads initial values of the scan condition parameters from the internal storage circuitry 18 as an examination is started (Step S101), and starts scanning according to the loaded initial values (Step S102). When this is done, an operator adjusts settings of the scan condition parameters as appropriate, while operating the ultrasound probe 1 and looking at an ultrasound image displayed on the monitor 2.

The control circuitry 17 determines whether index control has been received by the operator operating the button switch 3b (Step S103), and if it is determined that index control has been received (Step S103: Yes), the control circuitry 17 further determines whether or not the index is “0” (Step S104). If the index is “0” (Step S104: Yes), the control circuitry 17 refers to the index control table, and controls the transmitting and receiving circuitry 11 to transmit and receive ultrasound waves with “scan range: 100%” and “number of transmission and receptions: n” (n=“2”). Scanning is then performed under these scan conditions by the transmitting and receiving circuitry 11 (Step S105).

If the index is not “0” (Step S104: No), the control circuitry 17 subsequently determines whether or not the index is “1”, similarly (Step S106). If the index is “1” (Step S106: Yes), the control circuitry 17 refers to the index control table, and controls the transmitting and receiving circuitry 11 to transmit and receive ultrasound waves with “scan range: 50%” and “number of transmission and receptions: 2n” (n=“2”). Scanning is then performed under these scan conditions by the transmitting and receiving circuitry 11 (Step S107).

If the index is not “1” (Step S106: No), the control circuitry 17 subsequently determines whether or not the index is “2”, similarly (Step S108). If the index is “2” (Step S108: Yes), the control circuitry 17 refers to the index control table, and controls the transmitting and receiving circuitry 11 to transmit and receive ultrasound waves with “scan range: 33%” and “number of transmission and receptions: 3n” (n=“2”). Scanning is then performed under these scan conditions by the transmitting and receiving circuitry 11 (Step S109).

Further, if the index is not “2” (Step S108: No), the control circuitry 17 refers to the index control table, and controls the transmitting and receiving circuitry 11 to transmit and receive ultrasound waves with “scan range: 25%” and “number of transmission and receptions: 4n” (n=“2”). Scanning is then performed under these scan conditions by the transmitting and receiving circuitry 11 (Step S110).

The control circuitry 17 repeats the above described determination every time index control is received, and controls transmission and reception of ultrasound waves by the transmitting and receiving circuitry 11 according to the index set by the operator via the operating device 3.

For example, FIG. 6 illustrates a flow of this operation by the operator and ultrasound images (I1, I2, and I3) displayed on the monitor 2, together. Further, FIG. 6 illustrates ultrasound images obtained when a liver part of the subject P is scanned. Further, in the first embodiment, the control circuitry 17 further displays, on the monitor 2, parameter marks visually expressing the parameter values of the scan condition parameters being set.

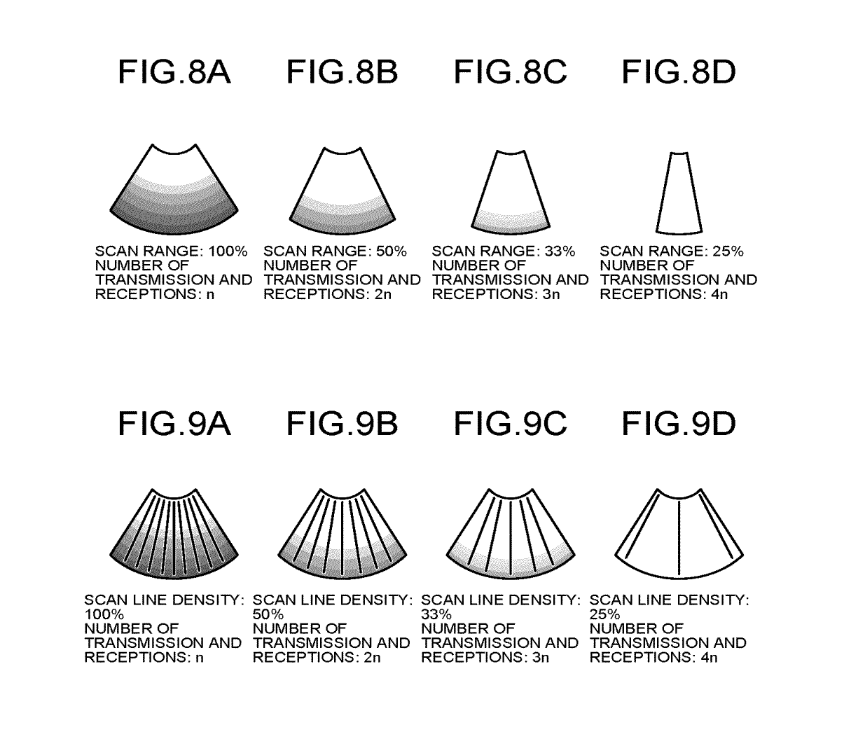

FIGS. 8A to 8D are diagrams illustrating the parameter marks in the first embodiment. FIG. 8A corresponds to the index, “0”, illustrated in FIG. 5, FIG. 8B to the index, “1”, FIG. 8C to the index, “2”, and FIG. 8D to the index, “3”. The parameter marks are simply aimed to make an operator to intuitively recognize the parameter values of the scan condition parameters being set, and thus their expression is not required to be exact. For example, it is sufficient if the manner in which the sensitivity of the deep part is improved as the field width is narrowed is expressed as illustrated in FIGS. 8A to 8D.

By displaying such parameter marks, an operator is able to recognize characteristics of the parameter values that the operator is trying to set (relations with the field width, spatial resolution, time resolution, sensitivity, and the like) just at first sight. It is considered that the operator will perform setting of the parameters while looking at the ultrasound image, and thus in the first embodiment, an example in which the parameter mark is also displayed on the monitor 2 is described, but the embodiment is not limited to this example. The parameter mark may or may not be displayed on the TCS 3a. A configuration in which display and non-display thereof are able to be switched over may be adopted also.

In the ultrasound images illustrated in FIG. 6, the diaphragm, blood vessels in the liver, and the like are visualized. If the index is “0”, as illustrated with a parameter mark M1 in FIG. 6, scanning is performed under the situation where the field width is sufficient but the sensitivity (in particular, the sensitivity of the deep part) is low. As illustrated with the ultrasound image I1, for example, a noise component is then generated in the deep part and visibility of the blood vessel image of the deep part is reduced. Further, for example, visualization ability for a part of the blood vessels in the liver, the part being surrounded by a dotted circle, is also reduced.

If the index is “1”, as illustrated with a parameter mark M2 in FIG. 6, scanning is performed under the situation where the field width is halved but the sensitivity is a little improved. When that is done, as illustrated with the ultrasound image I2, for example, the noise component of the deep part is a little reduced and visibility of the blood vessel image of the deep part is improved, but the blood vessel in the part surrounded by the dotted circle are still not visualized sufficiently.

In contrast, if the index is “3”, as illustrated with a parameter mark M3 in FIG. 6, scanning is performed under the situation where the field width is narrowed to ¼ but the sensitivity is considerably improved. As illustrated with the ultrasound image I3, for example, the noise component of the deep part is then resolved, the visibility of the blood vessel image of the deep part is improved, and the blood vessel in the part surrounded by the dotted circle is also visualized. That is, it is understood that as the number of transmission and receptions per scan line is increased, while the luminance of the biological image is maintained or improved, the luminance of the noise component of the deep part is reduced and visibility of the blood vessel image of the deep part is improved.

As described above, for example, for examination of a tumor in a liver part, since the liver is an organ with little motion in contrast to the heart, improvement of the sensitivity may be desired, rather than improvement of the time resolution. Practically, a tumor in a liver part is often present in a deep part of an ultrasound image and in such a case, scanning is desired to be performed with the sensitivity for the deep part in particular being improved. In this respect, in the first embodiment, by simple operation, with the spatial resolution being maintained, the sensitivity is also able to be improved. The index control table, the operation UI 3c and button switch 3b assigned with the index, the parameter marks and ultrasound images displayed on the monitor 2, and the like are just an example, and any of them may be arbitrarily modified. The same applies to the other embodiments.

Further, in the first embodiment, it has been described that control is performed with the index control table being prepared beforehand, but the embodiment is not limited to this. The control circuitry 17 may calculate a parameter value of another parameter on the spot according to a parameter value set for a certain parameter, and control transmission and reception of ultrasound waves with the calculated parameter value. The same applies to the other embodiments.

As described above, in the first embodiment, setting of a trade-off is able to be realized, with “number of transmission and receptions”, in addition to “scan range”, “scan line density”, and “frame rate”, being the scan condition parameters. As a result, scan condition parameters are able to be provided variously and an operator is able to flexibly change field width, spatial resolution, time resolution, sensitivity, and the like of ultrasound images.

### Modification of First Embodiment

In the above described first embodiment, the example, in which the trade-off between “scan range” and “number of transmission and receptions” is realized with “scan line density” and “frame rate” being fixed, has been described, but the embodiment is not limited to this example. For example, similar application may be made to an example, in which a trade-off between “scan line density” and “number of transmission and receptions” is realized with “scan range” and “frame rate” being fixed, an example, in which a trade-off between “frame rate” and “number of transmission and receptions” is realized with “scan range” and “scan line density” being fixed, and the like.

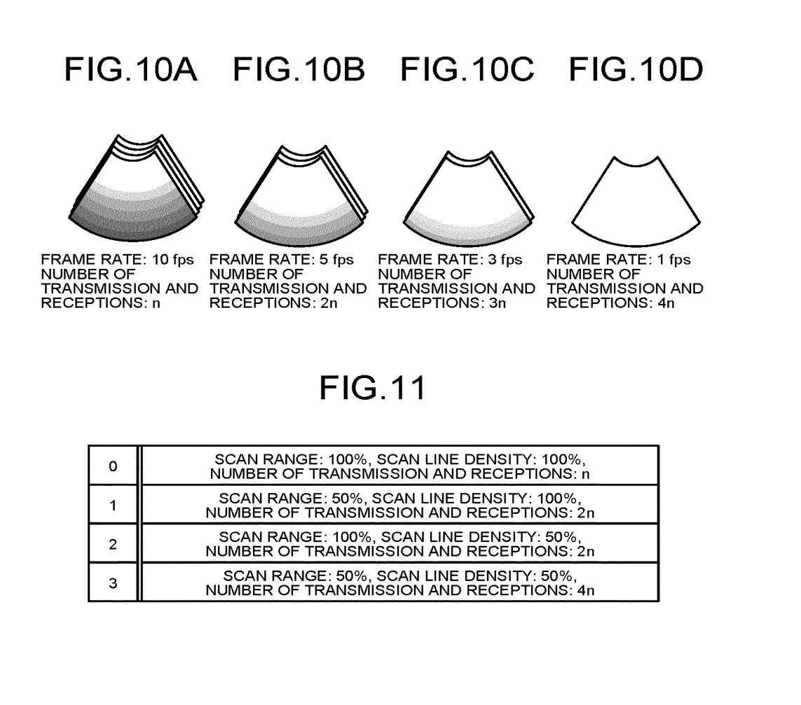

FIGS. 9A to 9D and FIGS. 10A to 10D are diagrams illustrating parameter marks in a modification of the first embodiment. For example, when a trade-off between “scan line density” and “number of transmission and receptions” is realized with “scan range” and “frame rate” being fixed, the control circuitry 17, for example, may prepare indices of combinations illustrated in FIGS. 9A to 9D and control transmission and reception of ultrasound waves while displaying parameter marks illustrated in FIGS. 9A to 9D on the monitor 2. In FIGS. 9A to 9D, the manner in which the scan line density is gradually decreased is visually expressed by the number of scan lines being decreased.

Further, for example, if a trade-off between “frame rate” and “number of transmission and receptions” is realized with “scan range” and “scan line density” being fixed, the control circuitry 17, for example, may prepare indices of combinations illustrated in FIGS. 10A to 10D and control transmission and reception of ultrasound waves while displaying parameter marks illustrated in FIGS. 10A to 10D on the monitor 2. In FIGS. 10A to 10D, the manner in which the frame rate is gradually decreased is visually expressed by the number of overlapped frames being decreased. The frame rate may be separately displayed with a numerical value, like “10 fps”, on the monitor 2 instead.

Further, in the above described first embodiment, the example, in which the trade-off between the two parameters of the four parameters is realized, has been described, but the embodiment is not limited to this example. The number of parameters that are able to be controlled by a trade-off may be increased further, to three parameters, four parameters, or the like. The combination of parameters may be arbitrarily modified also.

FIG. 11 is a diagram illustrating an index control table in the modification of the first embodiment. For example, the control circuitry 17 stores the index control table illustrated in FIG. 11. As illustrated in FIG. 11, for example, in the index control table, correspondingly with the index, “0”, “scan range: 100%”, “scan line density: 100%”, and “number of transmission and receptions: n” are stored. Herein, n=“2”. In the index control table, further, correspondingly with each of the indices, “1”, “2”, and “3”, “scan range”, “scan line density”, and “number of transmissions and receptions” are stored.

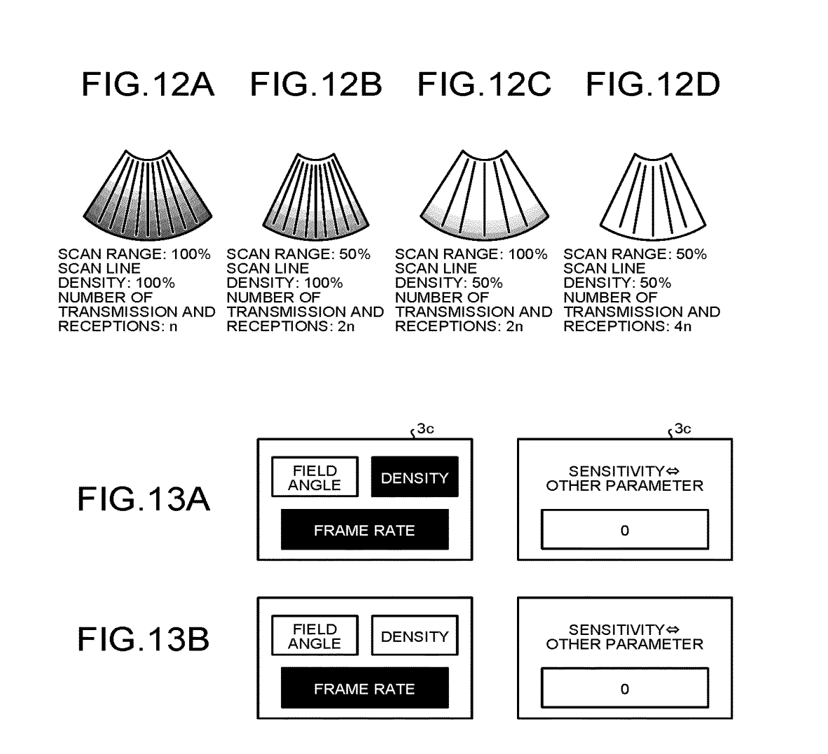

Further, FIGS. 12A to 12D are diagrams illustrating parameter marks corresponding to the index control table illustrated in FIG. 11. For example, the parameter marks illustrated in FIGS. 12A to 12D visually express information corresponding to the three parameters.

### Second Embodiment

In the above described first embodiment, the example, in which realization is achieved by provision of the operation UI of index control, has been described, but the embodiment is not limited to this example. In the second embodiment, an example will be described, in which index control is performed and an operation UI with a priority mode switch is provided. The ultrasound diagnostic apparatus 100 according to the second embodiment basically has the same configuration as that of the first embodiment, unless mentioned particularly.

For example, in the second embodiment, the control circuitry 17 displays, on the TCS 3a, the operation UI 3c for controlling at least one parameter of four parameters. Further, the control circuitry 17 displays, on the TCS 3a, the operation UI 3c for setting any one or more of the remaining parameters in a priority mode.

FIGS. 13A and 13B are diagrams illustrating the operation UI with the priority mode switch in the second embodiment. In FIGS. 13A and 13B, the operation UI 3c on the left is the operation UI 3c for setting any one or more of the three parameters, “scan range” (“field angle” in FIGS. 13A and 13B), “scan line density” (“density” in FIGS. 13A and 13B), and “frame rate”, in a priority mode. In FIGS. 13A and 13B, black and white are reversely expressed for any parameter set in the priority mode (with the background expressed in black and the text in white).

In FIG. 13A, “scan line density” and “frame rate” are set in the priority mode. In this case, the operation UI 3c on the right will automatically realize a trade-off between “number of transmission and receptions” (“sensitivity” in FIGS. 13A and 13B) and the remaining parameter, “scan range”. In other words, “scan line density” and “frame rate” set in the priority mode are maintained at high level, without being adjusted by a trade-off relation. This means that the same index control as the index control described with use of FIG. 5 in the first embodiment is performed, for example.

Further, in FIG. 13B, “frame rate” is set in the priority mode. In this case, the operation UI 3c on the right will automatically realize a trade-off between “number of transmission and receptions” and the remaining parameters, “scan range” and “scan line density”. In other words, “frame rate” set in the priority mode is maintained at high level, without being adjusted by a trade-off relation. This means that the same index control as the index control described with use of FIG. 11 in the first embodiment is performed, for example. The above described operation UI 3c is just an example, and the above described setting of the priority mode may be realized by another operation UI 3c. For example, combinations conceivable as combinations of parameters to be prioritized may be listed, and an operation UI 3c for causing a desired combination to be selected from the list may be provided.

As described above, in the second embodiment, by receiving setting of a parameter or parameters to be maintained at high level from the four parameters, a combination of parameters to be subjected to a trade-off is able to be flexibly changed according to the setting. Therefore, an operator is able to select the parameter or parameters to be fixed at will and to perform setting of the trade-off.

### Third Embodiment

In a third embodiment, provision of an operation UI 3c will be described for a case where a parameter, “number of transmission and receptions”, is newly incorporated into an existing ultrasound diagnostic apparatus 100 in which the parameter, “number of transmission and receptions”, has not been incorporated as a parameter adjustable by a trade-off. The ultrasound diagnostic apparatus 100 according to the third embodiment basically has the same configuration as that of the first embodiment, unless mentioned particularly.

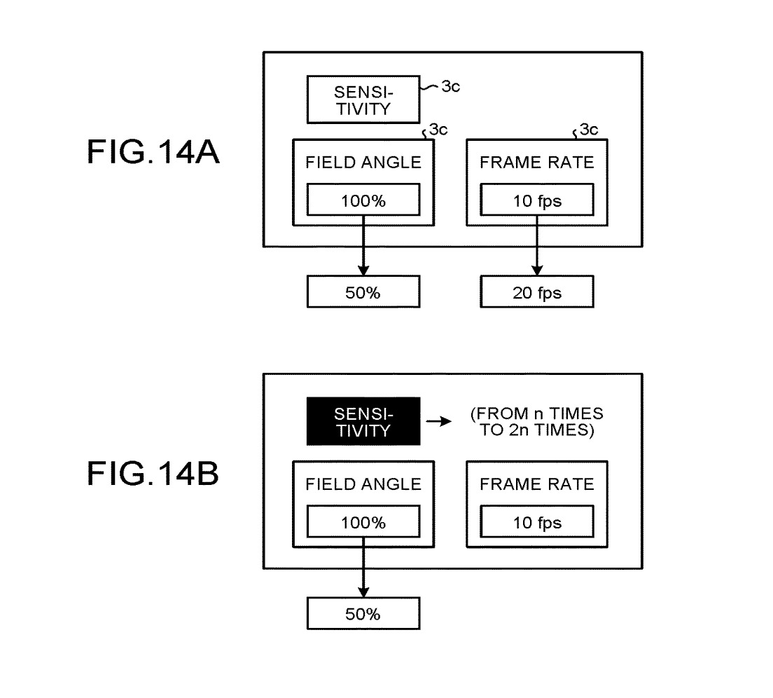

FIGS. 14A and 14B are diagrams illustrating an operation UI with a sensitivity ON/OFF switch in the third embodiment. For example, it is assumed that in the existing ultrasound diagnostic apparatus 100, the operation UI 3c realizing a trade-off between “scan range” and “frame rate” is being displayed on the TCS 3a. For example, as illustrated in FIG. 14A, it is set up such that the operation UI 3c for “field angle” indicating “scan range” and the operation UI 3c indicating “frame rate” are displayed, and when one of the parameter values is changed by an operator, the other parameter value in a trade-off relation with the one of the parameter values is automatically calculated and changed.

To such existing operation UIs 3c, for example, the control circuitry 17 provides an operation UI with a sensitivity ON/OFF switch for controlling ON/OFF of “number of transmission and receptions” (“sensitivity”) as illustrated in FIGS. 14A and 14B. In FIGS. 14A and 14B, if “number of transmission and receptions” is set ON, black and white are reversely expressed (with the background expressed in black and the text in white).

For example, as illustrated in FIG. 14A, if an operation to change “scan range” (“field angle”) from “100%” to “50%” is performed when “number of transmission and receptions” has been set OFF, a parameter value of “frame rate” is automatically calculated and “frame rate” is doubled to “20 fps” from “10 fps”. It is set such that the time resolution is improved correspondingly with the narrowing of the field width.

On the contrary, if an operation to change “scan range” (“field angle”) from “100%” to “50%” is performed when “number of transmission and receptions” has been set OFF as illustrated in FIG. 14B, a parameter value of “number of transmission and receptions” is automatically calculated to be doubled to “twice” from “n times”, without “frame rate” being changed. It is set such that the sensitivity is improved correspondingly with the narrowing of the field width.

The above described example is just an example. An operation UI for controlling ON/OFF of a parameter value of “number of transmission and receptions” may be set for an operation UI for adjusting other parameter values. Further, a display mode of the operation UI 3c may also be modified to any display mode, not being limited to the example illustrated in FIGS. 14A and 14B.

As described above, in the third embodiment, the parameter, “number of transmission and receptions”, is able to be newly incorporated into the existing ultrasound diagnostic apparatus 100, and thus even for an operator familiar with the existing operability, setting of “number of transmission and receptions” is able to be incorporated therein without discomfort.

### Fourth Embodiment

Subsequently, in a fourth embodiment, control of scan condition parameters is performed in association with specification of an ROI. For example, when color Doppler imaging (CDT) is performed, a region of interest may be specified on an ultrasound image by an operator. In this case, “scan range” usually just needs to be a range including the ROI, and thus in the fourth embodiment “scan range” is narrowed to the range including the ROI and according to this “scan range”, the above described control of the other parameters is performed. The ultrasound diagnostic apparatus 100 according to the fourth embodiment basically has the same configuration as that of the first embodiment, unless mentioned particularly.

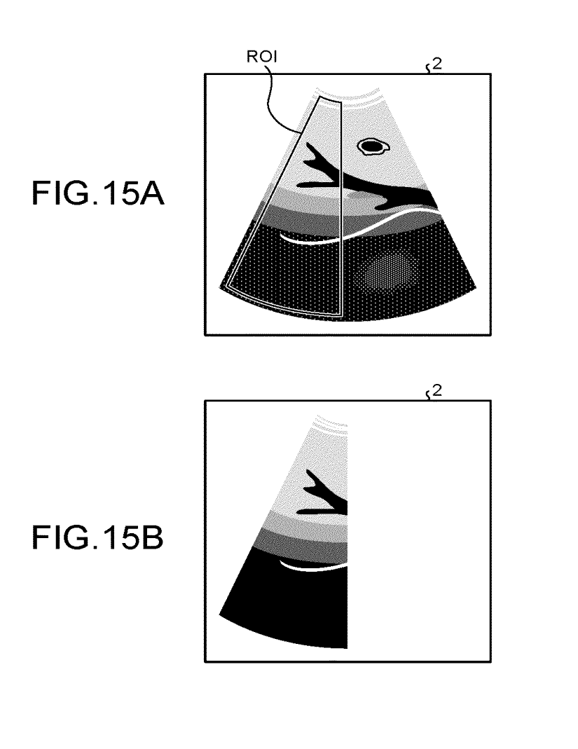

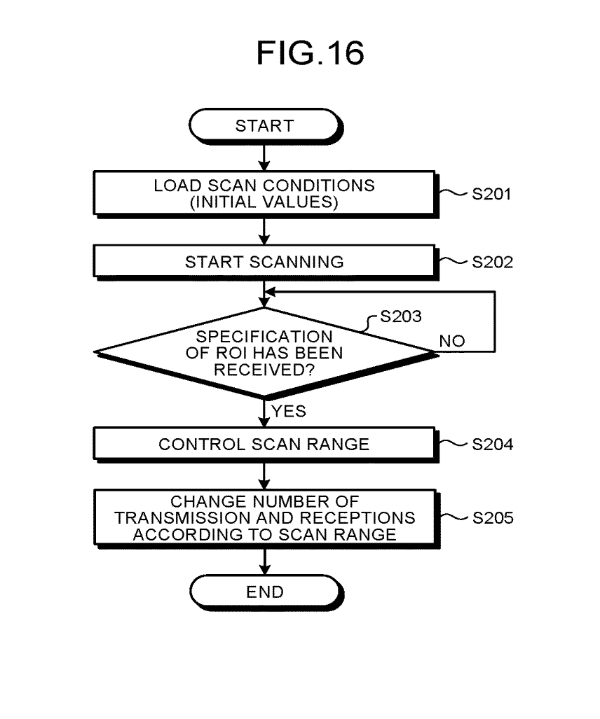

FIGS. 15A and 15B are diagrams for description of specification of an ROI in the fourth embodiment, and FIG. 16 is a diagram illustrating a processing sequence of parameter control in the fourth embodiment. For example, similarly to the first embodiment, it is assumed that a trade-off between “scan range” and “number of transmission and receptions” is determined beforehand to be realized with “scan line density” and “frame rate” being fixed. In this case, if an operator inputs specification of an ROI on an ultrasound image as illustrated in FIG. 15A, “scan range” is automatically narrowed to a range including the ROI and “number of transmission and receptions” is automatically increased, improving sensitivity of the ultrasound image, as illustrated in FIG. 15B.

That is, as illustrated in FIG. 16, the control circuitry 17 loads initial values of scan condition parameters from the internal storage circuitry 18 as an examination is started (Step S201), and starts scanning according to the loaded initial values (Step S202). Subsequently, if it is determined that specification of an ROI has been received (Step S203: Yes), the control circuitry 17 controls the transmitting and receiving circuitry 11 to narrow “scan range” up to a range including the ROI (Step S204). The control circuitry 17 then changes “number of transmission and receptions” according to the narrowed “scan range” and controls the transmitting and receiving circuitry 11 to transmit and receive ultrasound waves with the changed “number of transmission and receptions” (Step S205).

### First Modification of Fourth Embodiment

Association between parameter control and ROI specification is not limited to the above described embodiment. Although the example, in which the control circuitry 17 automatically increases “number of transmission and receptions” when “scan range” is narrowed according to specification of an ROI, has been described in the above described embodiment, the increase is not necessarily performed automatically. For example, the control circuitry 17 may display, on the TCS 3a, the operation UI 3c with the sensitivity ON/OFF switch as described in the third embodiment and may select whether or not to increase “number of transmission and receptions” according to whether or not “number of transmission and receptions” has been set ON.

If “scan range” is narrowed according to specification of an ROI when “number of transmission and receptions” has been set ON beforehand, the control circuitry 17 automatically increases “number of transmission and receptions”. On the contrary, even if “scan range” is narrowed according to specification of an ROI when “number of transmission and receptions” has been set OFF beforehand, the control circuitry 17 simply narrows “scan range” without automatically increasing “number of transmission and receptions”. The control circuitry 17 increases “number of transmission and receptions” when “number of transmission and receptions” is set ON thereafter.

The example, in which the trade-off between “scan range” and “number of transmission and receptions” is realized with “scan line density” and “frame rate” being fixed, has been described above. That is, although the example, in which the sensitivity is changed under the constraint where the spatial resolution and time resolution are constant, has been described, the embodiment is not limited to this example. For example, any one or both of “scan line density” and “frame rate” may be included further in the target of the trade-off.

### Second Modification of Fourth Embodiment

The setting of the priority mode described in the second embodiment may be combined further. For example, it is assumed that “scan range” is a range exceeding 50%, where “scan range” is normally 100%, when “scan range” is narrowed to a range including an ROI. In such a case, although it is difficult to increase “number of transmission and receptions”, the control circuitry 17 changes any one or both of “scan line density” and “frame rate”, instead of “number of transmission and receptions”.

For example, the control circuitry 17 displays, on the TCS 3a, the operation UI 3c for setting any one or both of “scan line density” and “frame rate” in the priority mode as exemplified in the second embodiment. If the control circuitry 17 determines that “number of transmission and receptions” is unable to be increased as a result of the ROI being specified by an operator and “scan range” being narrowed, the control circuitry 17 subsequently determines which parameter of “scan line density” and “frame rate” has been set in the priority mode.

If “scan line density” has been set in the priority mode, the control circuitry 17 calculates “scan line density” in an attempt to increase “scan line density”, such that “frame rate” is maintained, that is, without changing the total number of transmission and receptions required in generating ultrasound images of one frame. The control circuitry 17 then controls the transmitting and receiving circuitry 11 to transmit and receive ultrasound waves with the calculated “scan line density”.

Further, if “frame rate” has been set in the priority mode, the control circuitry 17 calculates “frame rate” in an attempt to increase “frame rate”, such that “scan line density” is maintained. The control circuitry 17 then controls the transmitting and receiving circuitry 11 to transmit and receive ultrasound waves with the calculated “frame rate”. As described above, the control circuitry 17 selects, according to the priority mode set by the operator, whether to redirect influence to “scan line density” or to “frame rate”, the influence being caused by transmission and reception of ultrasound waves corresponding to the deviation of “scan range” from normal “scan range”.

### Third Modification of Fourth Embodiment

Further, the example, in which specification of an ROI is received first and “scan range” is next narrowed to a range including the ROI, has been described in the above described embodiment, but the embodiment is not limited to this example. For example, setting for narrowing “scan range” may be done first, and thereafter, an operator may specify an ROT in the narrowed “scan range”.

As described above, according to the fourth embodiment, since the control of the scan condition parameters is performed according to specification of an ROI, the specification of the ROI and the control of “scan range”, and control of the other parameters according thereto, are able to be performed more easily.

### Other Embodiments

Although various embodiments have been described above, the embodiments are not limited to them. For example, what has been described in the respective embodiments may be combined with one another, and may be arbitrarily modified by switching over the exemplified parameters, combining another parameter (for example, sound pressure, or the like) further therewith, and so on. Further, the operation UI is not limited to the operation UIs described in the respective embodiments. Any mode of the operation UI, for example, a mode, in which functions are assigned only to software switches, or a mode, in which functions are assigned only to hardware operating devices, may be adopted.

B-mode and Other Modes

Further, the case where the operation is performed in the harmonic imaging mode has been described in the above described embodiments, but the embodiments are not limited to this case. For example, application may be made similarly to a case where operation is performed in the normal B-mode, a case where operation is performed in the CDI (in particular, TDI) mode, or the like. In that case, the ultrasound diagnostic apparatus includes transmitting and receiving circuitry, adding circuitry, image generating circuitry, and control circuitry. The transmitting and receiving circuitry performs, more than once on the same scan line, an ultrasound transmission and reception in order to receive reflected wave data required in generating an image according to the number set as a scan condition parameter. The adding circuitry adds together the reflected wave data received as a result of the ultrasound transmission and receptions. The image generating circuitry generates an image by using the added reflected wave data. The control circuitry controls the transmitting and receiving circuitry, based on a relation between the number of the ultrasound transmission and receptions and a scan condition parameter other than the number. Further, not being limited to the case where the operation is performed by the harmonic imaging of the polarity determination technique, similar application may be made to a case where operation is performed by harmonic imaging of a filter method or the like.

Program

Further, instructions indicated in the processing sequences illustrated in the above described embodiments may be executed based on a program, which is software. The same effects as the effects by the ultrasound diagnostic apparatus 100 of the above described embodiments may be obtained by a general purpose computer storing this program beforehand and loading this program. The instructions described in the above embodiments are recorded, as the program that is executable by the computer, in a magnetic disk (a flexible disk, a hard disk, or the like), an optical disk (a CD-ROM, a CD-R, a CD-RW, a DVD-ROM, a DVD±R, a DVD±RW, or the like), a semiconductor memory, or a recording medium similar thereto. A storage format of the recording medium may be of any mode as long as the recording medium is readable by the computer or an embedded system. The computer is able to realize the same operation as that of the ultrasound diagnostic apparatus 100 of the above described embodiments by loading the program from this recording medium and executing the instructions described in the program by a central processing unit (CPU) based on this program. Further, when the computer obtains or loads the program, the computer may obtain or load the program through a network.

According to the ultrasound diagnostic apparatus of at least one of the above described embodiments, scan condition parameters are able to be provided variously.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnostic apparatus, comprising:
transmitting and receiving circuitry configured to perform, more than once on the same scan line, an ultrasound transmission and reception repeatedly performed with phase polarities being inverted on the same scan line, according to a number set as a scan condition parameter;
adding circuitry configured to add together reflected wave data received as a result of the ultrasound transmission and receptions;
image generating circuitry configured to generate an image by using the reflected wave data that have been added together; and
control circuitry configured to control the transmitting and receiving circuitry, based on a relation between the number of the ultrasound transmission and receptions and a scan condition parameter other than the number.

2. The ultrasound diagnostic apparatus according to claim 1, wherein the control circuitry controls the transmitting and receiving circuitry, based on a trade-off relation between the number of the ultrasound transmission and receptions and the scan condition parameter other than the number.

3. The ultrasound diagnostic apparatus according to claim 1, wherein the control circuitry controls the transmitting and receiving circuitry, based on the relation between the number of the ultrasound transmission and receptions and the scan condition parameter other than the number, the scan condition parameter being at least one of scan range, scan line density, and frame rate.

4. The ultrasound diagnostic apparatus according to claim 1, further comprising:
a user interface (UI) configured to enable adjustment of the relation between the number of the ultrasound transmission and receptions and the scan condition parameter other than the number is included, wherein
the control circuitry receives an input of a setting with respect to the UI.

5. The ultrasound diagnostic apparatus according to claim 4, further comprising:
a table configured to store list of combinations of the numbers of the ultrasound transmission and receptions and parameter values of the scan condition parameter other than the number correspondingly with indices, wherein
the UI receives a selection of an index, and
the control circuitry receives an input of the selection with respect to the UI.

6. The ultrasound diagnostic apparatus according to claim 4, wherein
the UI receives a setting for maintaining without change a parameter value of its own parameter when a parameter value of the number is changed in relation to the scan condition parameter other than the number of the ultrasound transmission and receptions, and
the control circuitry receives an input of a selection with respect to the UI.

7. The ultrasound diagnostic apparatus according to claim 4, wherein the control circuitry displays the UI on a second display that is different from a first display that displays an ultrasound image.

8. The ultrasound diagnostic apparatus according to claim 1, wherein the control circuitry changes a scan range according to a specified region of interest when specification of the region of interest on an ultrasound image is received, and controls the transmitting and receiving circuitry with the number of the ultrasound transmission and receptions derived based on a trade-off relation with the changed scan range.

9. The ultrasound diagnostic apparatus according to claim 1, wherein the control circuitry displays together a mark visually expressing a relation between the number of the ultrasound transmission and receptions and the scan condition parameter other than the number, on a display that displays an ultrasound image.

10. The ultrasound diagnostic apparatus according to claim 1, wherein
the transmitting and receiving circuitry performs, on the same scan line, one set or plural sets of one-set ultrasound transmission and reception/receptions repeatedly performed with phase polarities inverted, and
the control circuitry controls the transmitting and receiving circuitry and switches over between the one set of ultrasound transmission and reception and the plural sets of ultrasound transmission and receptions.

11. An ultrasound diagnostic apparatus, comprising:
transmitting and receiving circuitry configured to perform, more than once on the same scan line, an ultrasound transmission and reception in order to receive reflected wave data required in generating an image according to the number set as a scan condition parameter;
adding circuitry configured to add together the reflected wave data received as a result of the ultrasound transmission and receptions;
image generating circuitry configured to generate an image by using the reflected wave data that have been added together; and
control circuitry configured to control the transmitting and receiving circuitry, based on a relation between the number of the ultrasound transmission and receptions and a scan condition parameter other than the number.
